(19) United States
(12) Patent Application Publication
(10) Pub. No.: US 2026/0338393 A1
(43) Pub. Date: Oct. 29, 2026

(54) IMPROVED REMOTELY OR AUTONOMOUSLY OPERABLE AIR DISINFECTION SYSTEM

(71) Applicant: AKARA ROBOTICS LIMITED, Dublin (IE)

(72) Inventors: Conor McGinn, Dublin (IE); Eamonn Bourke, Dublin (IE); Michael Cullinan, Dublin (IE)

(73) Assignee: AKARA ROBOTICS LIMITED, Dublin (IE)

(21) Appl. No.: 18/877,665

(22) PCT Filed: Jun. 20, 2023
(86) PCT No.: PCT/EP2023/066707

§ 371 Date: Dec. 20, 2024

(30) Foreign Application Priority Data
Jun. 20, 2022 (GB) 2209024.5

(51) Int. Cl. F24F 7/003; A61L 9/16; A61L 9/20; B01D 46/00; B01D 46/44; B01D 46/46; F24F 8/10; F24F 8/22; F24F 13/10; F24F 110/12; F24F 110/22; F24F 110/52; F24F 110/64; F24F 110/66; F24F 110/70; F24F 120/14; G05D 1/648

(52) U.S. Cl. CPC F24F 7/003; A61L 9/16; A61L 9/20; B01D 46/0028; B01D 46/0045; B01D 46/442; B01D 46/448; B01D 46/46; F24F 8/10; F24F 8/22; F24F 13/10; G05D 1/648; A61L 2209/111; A61L 2209/12; A61L 2209/14; A61L 2209/15; A61L 2209/16; B01D 2273/30; B01D 2279/35; B01D 2279/50; B01D 2279/65; F24F 2110/12; F24F 2110/22; F24F 2110/52; F24F 2110/64; F24F 2110/66; F24F 2110/70; F24F 2120/14

(57) ABSTRACT

An air disinfection system for the treatment of air containing contaminants, in one or more enclosed spaces. The system has an enclosed space ventilation system with air quality sensors in the enclosed space, ventilation devices for providing ventilation in the enclosed space and a communications interface operatively connectable to an air disinfection apparatus and the one or more contaminant sensor. The system also includes the air disinfection apparatus including a treatment system with an extractor for drawing air in from the enclosed space and a decontamination system which receives the air from the extractor, the decontamination system to remove and/or neutralize the contaminant aerosols in the air, an outlet for expelling treated air from the device and a moveable support to which the treatment system is attached. The air disinfection system has a system controller for controlling the operation of the air disinfection apparatus and the enclosed space ventilation system.

### FIELD

The present disclosure relates to a system for air disinfection or decontamination. In particular, the present disclosure relates to an interconnected system comprising one or more air disinfection devices that can be autonomous, semi-autonomous or remotely controlled.

### BACKGROUND

Airborne transmission is the spread of infectious microorganisms through aerosol droplets produced when an infected person breathes, coughs, sneezes or talks. These droplets can land in the mouths or noses of people who are nearby or possibly be inhaled into the lungs. Spread is more likely when people are in close contact within about 1.8 metres (m). Airborne transmission of COVID-19 has also been shown to occur through aerosols containing residual parts of droplets, that are able to stay suspended in the air for longer periods of time.

Transmission is much more likely to occur at indoor locations because the air is less able to flow and is mostly contained in the enclosed space. Transmission also occurs in healthcare settings, often when aerosol-generating medical procedures are performed on patients. Long distance dispersal of pathogenic particles has been detected in ventilation systems of a hospital, indicating the possibility of long-range aerosol transmission. Other pathogens comprising viruses, moulds and bacteria may be present in both recirculated air and fresh external air and may be hazardous to health.

In the case of hospitals, certain medical procedures are defined as aerosol-generating events if they cause the patient to stimulate coughing and promote the generation of aerosols. Examples of aerosol-generating procedures (AGPs) listed by the WHO include tracheal intubation, non-invasive ventilation (e.g. BiLevel positive airway pressure, continuous positive airway pressure), tracheotomy, cardiopulmonary resuscitation, manual ventilation before intubation, bronchoscopy, sputum induction by using nebulized hypertonic saline, dentistry and autopsy procedures.

During AGPs, physicians are required to wear appropriate PPE including respirators. However, since the procedure leads to contamination of air in the room, a period of downtime is often mandated to ensure appropriate replenishment of the air in the room. The duration of downtime is typically proportional to the size of the room and the flow rate of the air ventilation system, and is normally in the region of 20-30 minutes for a replenishment at a 99% level.

However, these air replenishment times assume perfect mixing of the air, which is seldom experienced in practice and it has been proposed by some experts that the duration of time recommended based on this assumption should be multiplied by a mixing factor that will vary depending on the level of air stagnation in the room. It has been predicted that the actual time needed for 99% replenishment for rooms with more than 6 air changes per hour is likely to be three times the value predicted in the basic equation (i.e. mixing factor=3). With this adjustment, a room that previously required 30 minutes downtime to achieve a 99% air replenishment level would actually require 90 minutes to achieve the desired effect.

Building ventilation plays an important role in indoor air quality. This typically takes the form of forced ventilation, whereby outside air is extracted, filtered, heated or cooled as required and piped through ducting around the building. However, the volume of air that can be transported through building ventilation is limited, and often requires considerable energy to transport through the pipe system and heat/condition it. Furthermore, the locations of air vents are often suboptimal, meaning that the rate of air replenishment in parts of the room is contaminated can be slow.

An aerosol generating event may also simply be a person (or persons) in a room in any setting such as an office, clinic, entertainment venue or classroom.

A number of air purification and sterilisation solutions are known.

Static air filtration systems (including HEPA filtration and UV). However, they are rarely optimally positioned to provide aerosol extraction at source (being unable to adapt to the position of people in the environment) and therefore has very limited ability to constrain the spread of an aerosol at source.

Hydrogen Peroxide Vapour (HPV) and other room fogging technologies expel a gas, and do not have fans that extract air from a room. Many of these technologies require the room to be vacated during use.

U.S. Pat. No. 5,240,478A describes a mobile platform with filter, fan and UV lights for the treatment of air. There is no discussion of autonomous capabilities with the assumption that it is pushed into place.

U.S. Pat. No. 5,891,399A describes a mobile air disinfection method using various powered and unpowered means. The shell of the chamber can also be selectively moved to allow UV light to treat surfaces in the room.

CN111594952A, CN113805582A, CN213347056U and CN213362768U describe mobile autonomous air disinfection devices that use mechanical filters, UVC lamps, blowers, ground cleaning components and combinations thereof.

US2022111323A1 describe a mobile air disinfection device that uses a novel mechanism to maximise flowrate through the device.

When deployed in rooms, existing air purification devices tend to perform in isolation, as opposed to a coordinated system for air replenishment.

### SUMMARY OF THE INVENTION

It is an object of the invention, as set out in the appended claims, to overcome at least one of the above-referenced problems.

It is a further object of the present invention to shorten the period of downtime needed to replenish the air in a room following an aerosol generating event.

In accordance with a first aspect of the invention there is provided an air decontamination system for the treatment of air containing contaminants, in one or more enclosed spaces, the system comprising:
an enclosed space decontamination system comprising:
   one or more air quality sensor in the enclosed space;
   one or more decontamination device for treating air in the enclosed space; and
   a communications interface which is operatively connectable to an air decontamination apparatus and the one or more contaminant sensor,
an air decontamination apparatus comprising:
   a treatment system which has,
   an extractor for drawing air in from the enclosed space and
   a decontamination system which receives the air from the extractor, the decontamination system being adapted to remove and/or neutralise the contaminant aerosols in the air;
   an outlet for expelling treated air from the device;
   a moveable support to which the treatment system is attached, which is moveable across a surface in an autonomous or manual manner; and wherein
the air decontamination system has a system controller for controlling the operation of the air decontamination apparatus and the enclosed space decontamination system.

In accordance with a second aspect of the invention there is provided an air decontamination apparatus for the treatment of air containing contaminant aerosols, in an enclosed space, the apparatus comprising:
a treatment system which has, an extractor for drawing air in from the enclosed space and
a decontamination system which receives the air from the extractor, the decontamination system being adapted to remove and/or neutralise the contaminant aerosols in the air;
an outlet for expelling treated air from the device;
a moveable support to which the treatment system is attached, which is moveable across a surface; and
a controller for controlling the operation of the treatment system and the moveable support.

In at least one embodiment, the air decontamination system as claimed in claim 1 wherein, the system controller is located upon the air decontamination apparatus.

In at least one embodiment, the system controller is located within the enclosed space decontamination system.

In at least one embodiment, the air decontamination apparatus may operate in a series of enclosed spaces by connecting to the communications interface in the enclosed space such that the controller, when in communication with the enclosed space communication interface can adapt the air decontamination it provides in response to information received from the enclosed space communication interface.

In at least one embodiment, the air quality sensor comprises a network of sensors positioned in the enclosed space.

In at least one embodiment, the system controller uses a model of the enclosed space based on one or more of the following variables: room dimensions; air vent location; air flow; and time of day to provide optimised air decontamination for the room accounting for quality and energy efficiency

In at least one embodiment, the air quality sensors comprise one or more of CO₂, particulate matter, Nitrous oxide, volatile organic compounds, bioburden, detection of specific pathogens, temperature, humidity,

In at least one embodiment, the air quality sensors comprise occupancy, activity and other environmental parameters that influence the need for air replenishment due to their effect on air quality.

In at least one embodiment, enclosed space decontamination system comprises stationary air purifiers which comprise one or more blower, air filter, UV decontaminator and/or chemical air disinfector.

In at least one embodiment the system integrates with the forced ventilation system in a building within which the one or more enclosed space is located to coordinate an effective means to maintain air quality.

In at least one embodiment the system accounts for outdoor air quality, temperature, humidity. This could include, for example, relying more heavily on the system required here in preference to the buildings ventilation system if large changes in temperature/humidity are required to match desired room conditions.

In at least one embodiment, the contaminants are aerosols.

In at least one embodiment static air treatment units located within the enclosed space integrate with the air quality controller.

In at least one embodiment, the static air treatment units are mounted on the walls or ceiling or the floor.

In at least one embodiment, the static air treatment units use technology such as blowers, air filters (HEPA etc), radiation etc to disinfect air.

In at least one embodiment, the static air treatment units may further include air quality sensors which act as inputs to the overall system. These static air treatment units are controlled by the system controller, reacting to changes in air quality, activity etc based on sensor input and the room model.

In at least one embodiment, the extractor comprises a fan.

In at least one embodiment, the extractor is connected in series with one or more passive mechanical filters.

In at least one embodiment, the extractor comprises an inlet.

In at least one embodiment wherein at least one sensor is configured for determining the position of the device in the space.

In at least one embodiment, the inlet is configurable to selectively draw air into the device from a predetermined specific space around the device. The space to draw air from can be determined by input from the sensors in the environment, on the mobile base, direct input from an operator or any combination.

In at least one embodiment, the space may be defined by an angle to the horizontal around the device.

In at least one embodiment, the angle is 200 to 360 degrees.

In at least one embodiment, the angle is less than 90 degrees and preferably 60 degrees.

In at least one embodiment, the inlet can be extended from the mobile air decontamination device by means of a flexible tube and orientated to efficiently capture contaminated air from an aerosol generator.

In at least one embodiment, the outlet is controllable to set the direction in which the treated air is expelled from the device. This can decrease the likelihood of already treated air being recirculated through the device, improve the flow of air through the room as the air can be directed in a coordinated way and increase the mixing of air in the room as areas of stagnation can be disrupted.

In at least one embodiment, the decontamination system comprises a mechanical filter.

In at least one embodiment, the mechanical filter is a HEPA filter.

In at least one embodiment, the decontamination system further comprises a radiation source.

In at least one embodiment, the radiation source is a UV source.

In at least one embodiment, the radiation source is a UVC source.

In at least one embodiment, the radiation source is a far-UVC source.

In at least one embodiment, the radiation source is a plasma source.

In at least one embodiment, the sensors comprise a movement sensor.

In at least one embodiment, the sensors comprise a detector for detecting an aerosol generating person or object.

In at least one embodiment the sensors include air quality sensors (CO₂, particulate matter, Nitrous oxide, volatile organic compounds, bioburden, detection of specific pathogens, temperature, humidity, etc)

In at least one embodiment, the sensor comprises a LIDAR.

In at least one embodiment, the sensor comprises an ultrasonic proximity detector.

In at least one embodiment, the sensor comprises a depth camera.

In at least one embodiment, the controller comprises computing means for controlling the sensors and receiving sensor data.

In at least one embodiment, the controller comprises computer software for controlling the device.

In at least one embodiment, the software is configured to determine the optimal position of the device in the space.

In at least one embodiment, the software is configured to identify aerosol generating source(s) in the space.

In at least one embodiment the software is configured to plan and execute a navigation path from the device's current location to a location adjacent to the aerosol generating source.

In at least one embodiment, the software is configured to determine the optimal position in the room to position itself and direct the flow of exhaust air to optimize airflow.

In at least one embodiment, the software is configured to estimate the level of replenishment and provide a notification to the operator when the desired air replenishment has been obtained.

In at least one embodiment, the controller controls the movement of the apparatus in response to inputs from the navigation sensors.

In at least one embodiment, the moveable support comprises a base.

In at least one embodiment, the moveable support comprises wheels mounted for contact with a surface across which the apparatus can travel.

In at least one embodiment, the apparatus comprises a robotic system.

In at least one embodiment, the apparatus is an autonomous robotic system.

In at least one embodiment, the apparatus is semi-autonomous.

In at least one embodiment, the apparatus is remotely controlled.

In at least one embodiment, the apparatus is manually controlled.

In accordance with a third aspect of the invention there is provided an air decontamination apparatus for the treatment of air containing contaminant aerosols, in an enclosed space as defined herein and operable with the enclosed space decontamination system as defined in the appended claims.

### BRIEF DESCRIPTION OF THE FIGURES

The invention will be more clearly understood from the following description of an embodiment thereof, given by way of example only, with reference to the accompanying drawings, in which: FIG. 1 is a schematic view of an air decontamination apparatus;

FIG. 2a is a front perspective view of an air decontamination apparatus and FIG. 2b is a rear view of the same;

FIG. 3a is a schematic plan view of the directional inlet system where the inlet spans an angle of approximately 270 degrees surrounding the device, FIG. 3b is a schematic plan view of the directional inlet system where the inlet spans an angle of approximately 60 degrees surrounding the device and FIG. 3c is a schematic plan view of the device connected to an extraction system;

FIG. 4a is a perspective view of the directional inlet system where the inlet spans an angle of approximately 270 degrees surrounding the device, FIG. 4b is a perspective view of the directional inlet system where the inlet spans an angle of approximately 60 degrees surrounding the device and FIG. 4c is a perspective view of the device connected to an extraction system;

FIG. 5a is a front perspective view of an embodiment of the present invention which shows the directional exhaust system and FIG. 5b is a rear perspective view of an embodiment of the present invention which shows the directional exhaust system;

FIG. 6a is a schematic illustration of air exhalation by a person in an enclosed space and FIG. 6b is a schematic illustration of air exhalation by a person in an enclosed space using a device in accordance with the present invention;

FIG. 7a is a schematic illustration of air flow in an enclosed space with a traditional vent and FIG. 7b is an illustration of air flow in an enclosed space using a device in accordance with the present invention;

FIG. 8 is a schematic diagram of an example of a control system in accordance with the present invention;

FIG. 9 is a schematic diagram of the wider system, comprising of the air decontamination apparatus and other auxiliary sensors;

FIG. 10a is a schematic illustration of the system illustrated in FIG. 9 in which the mobile air decontamination apparatus, static air decontamination units and various auxiliary sensors collaborate and adapt to deliver better air quality, FIG. 10a illustrates the system in its normal operating state, FIG. 10b is an illustration of the system operating in a night mode or ECO state, FIG. 10c is an illustration of the system operating in a maximum air replenishment state, FIG. 10d is an illustration of the system operating in a clean air state, where the mobile air decontamination apparatus is positioned to act as a barrier for potentially contaminated air;

FIG. 11 is a schematic diagram illustrating the finite states in which the system can operate and to which it can transition;

FIG. 12 illustrates perspective views of the mobile air decontamination apparatus with air intake and exhaust positions inverted, as well as directional exhaust capabilities;

FIG. 13 illustrates perspective views of the mobile air decontamination apparatus with air intake towards the top and air exhaust towards the bottom of the apparatus, as well as directional exhaust capabilities;

FIG. 14 illustrates perspective views of the mobile air decontamination apparatus with air intake towards the top and air exhaust towards the bottom of the apparatus; and

FIG. 15 illustrates perspective views of the mobile air decontamination apparatus with air intake towards the bottom and air exhaust towards the top of the apparatus.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As referred to herein, the device of the present invention may be configured as a robot. A robot is a machine, especially one programmable by a computer, capable of carrying out a complex series of actions automatically. It can be guided by an external control device, or the control may be embedded within.

### Exemplification

In accordance with the present invention, the apparatus may be an autonomous, semi-autonomous or remotely controlled device including a robot.

FIG. 1 is a schematic view 1 of an air decontamination apparatus showing a basic decontamination process. The device comprises a housing 3 at the top of which is an inlet grill 5 with an antimicrobial coating which helps to inhibit the growth of microbes at the inlet. Below the inlet grill 5 and inside the housing, an intake fan 7 draws air through the inlet grill 5 from the device's surroundings. Air flow into and through the device is shown by reference numeral 13. The intake air contains aerosol particulates 15 and aerosol droplets 17.

The intake fan 7 draws air through a high-efficiency particulate absorbing (HEPA) filter 9, in this example an H13 HEPA filter, which removes up to 99.97% of airborne allergens and pollutants including dust, bacteria, mold, spores and smoke.

The air then passes through a carbon filter 10 which absorbs odours and gaseous pollutants and ultraviolet (UV) lamps 11 which destroys viruses or mold remaining in the air. A plasma lamp may also be used in place of the UV lamp.

The air outlet 19 has an antimicrobial coating which helps to inhibit the growth of microbes at the outlet and decontaminated air 21 exits the device at the bottom of the housing 3.

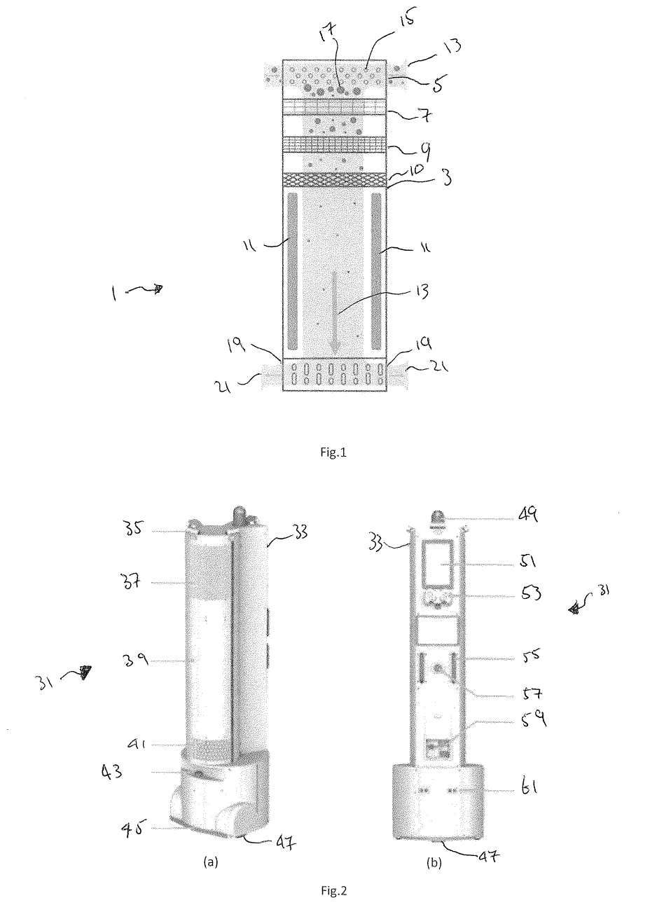

FIG. 2a is a front perspective view of an air decontamination apparatus in accordance with the present invention and FIG. 2b is a rear view of the same. The device 31 comprises a housing 33, a depth camera 35 positioned on the top of the housing 22. The depth camera 35 measures the range or distance of an object from the device. HEPA filter 37 removes most of the airborne allergens and pollutants including dust, bacteria, mold, spores and smoke and UVC lamps 39 neutralise any remaining mould or viruses. UVC is ultraviolet light in the wavelength range 200-280 nm. After treatment, the air exits from the device via outlet 41.

In this example, the device has a safety edge 45 and wheels 47 to allow the device to be moved around a space safely. A light detection and ranging (lidar) system 43, which also measures object range or distance, and a LED beacon 49 are provided in addition to the depth camera 35 in order to allow the position of the device to be detected. The light 49 is used as a visual indicator to determine whether the device is turned on and flashes to indicate the duration of time remaining in the decontamination procedure. The device may also comprises a voice control function, which allows the operator to control and interrogate the device without the requirement to physically touch a surface. In this example, the device further comprises a display screen 51, controller/joystick 53 for moving the device, an emergency stop button 57, power connector and data I/O ports 59 and an ultrasonic proximity detector 61.

The display screen 51 is positioned on the rear of the device and shows a representation of the space in which the device is to be used. The representation updates for each room where it is programmed to operate. In addition, the display is updated to instruct a human operator where the robot should be placed for optimal air mixing performance.

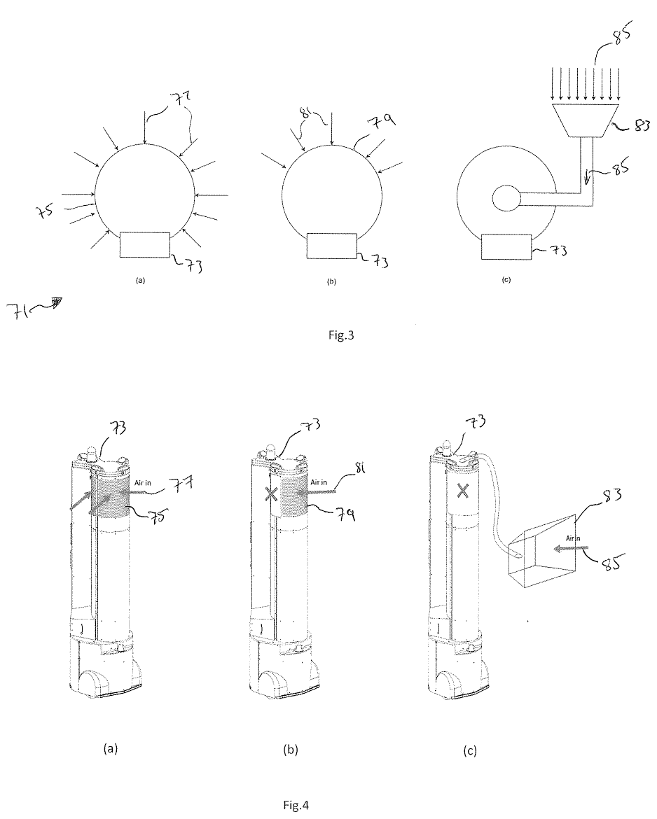

FIGS. 3 and 4 show examples of the manner in which air may be drawn into the device in different configurations.

FIG. 3a is a plan view of the directional inlet system. It shows a device with a housing 73 and an air inlet 75 which spans an angle of approximately 270 degrees surrounding the device to allow air 77 to be drawn into the device from most of the surrounding area. FIG. 4a is a perspective view of the directional inlet system as shown in FIG. 3a.

FIG. 3b is a plan view of the directional inlet system. It shows a device with a housing 73 and an air inlet 79 which spans an angle of approximately 60 degrees surrounding the device to allow air 81 to be drawn into the device from a smaller section of the surrounding area. In addition, the use of a smaller span angle will increase the speed of the air and the power per unit area to allow the fan to pull more air into the device and increase the distance from which air may be drawn into the device. FIG. 4b is a perspective view of the directional inlet system as shown in FIG. 3b.

FIG. 3c is a plan view of the directional inlet system. It shows a device with a housing 73 connected to an extraction system 83. Air 85 is drawn into the device via the extraction system 83. FIG. 4c is a perspective view of the directional inlet system as shown in FIG. 3c. Air extracted from a region outside the robot's local surroundings is passed through the device via the extractor 83 and flexible ducting.

FIG. 5a is a front perspective view of an embodiment of the present invention and FIG. 5b is a rear perspective view of the same. It shows the device 91 with an inlet 93. The direction of air flow is 95 is shown along with an air expulsion system 97.

The air expulsion system 97 provides a directional exhaust system in which air is drawn in from the front of the device's surroundings, processed through the device's filters and radiant sources, and expelled from the rear in a direction and at a velocity that can be actively controlled.

In at least one other embodiment, the optimal position of the device is determined by an artificial intelligence (AI) algorithm. In addition, the AI algorithms may perform any or all of the following tasks:
identifying aerosol generating source(s) in the room;
planning and executing a navigation path from the robot's current location to a location adjacent to the aerosol generating source;
determining the optimal position in the room to position itself and direct the flow of exhaust air to optimize airflow; and
estimating the level of replenishment and provide a notification to the operator when the desired air replenishment has been obtained.

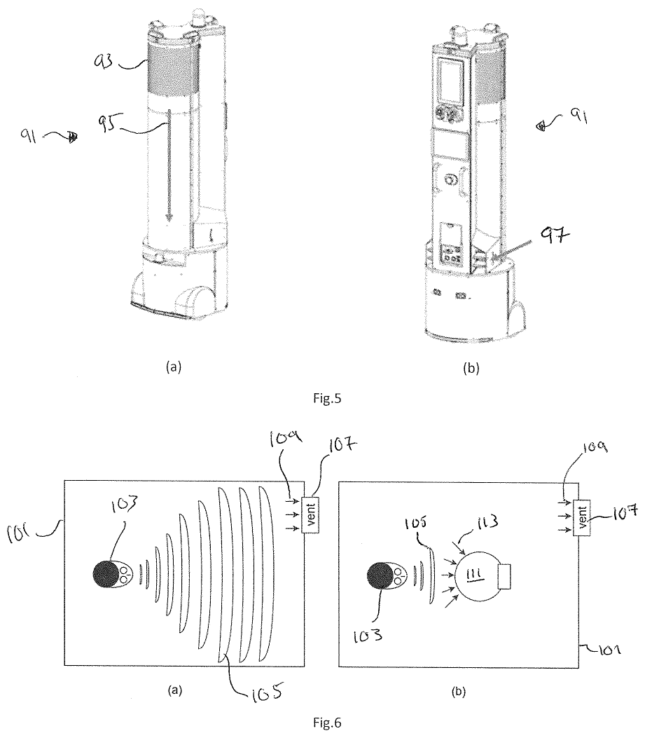

FIG. 6a is a schematic illustration of air exhalation by a person in an enclosed space and FIG. 6b is a schematic illustration of air exhalation by a person in an enclosed space using a device in accordance with the present invention. FIG. 6a shows an enclosed space 101, an occupant 103, aerosol generation by exhalation 105 by the occupant, a vent 107 and air flow to vent 109. FIG. 6b shows the space 101 when a device in accordance with the present invention is introduced into the space 101 at the commencement of the aerosol generating activity, with the device 111 being positioned adjacent to the source of aerosol generation. The inlet on the device 111 is collinear with the direction of aerosol generation 105. By positioning the device close to the source and configuring the inlet on the device to draw air from the direction of the aerosol source, for example as shown in FIG. 3b, the device acts as an extractor, and reduces the concentration of the contaminated aerosol in the room.

Under normal circumstances, aerosols from the person 105 will be projected throughout the room. Extraction vents 107, which are normally located on ceilings or walls, have a limited effect on constraining the spread of these aerosols. When the device is placed close to the source and the inlet configured to draw air in from the direction of the aerosol source, it acts as an extractor and decontaminates the air, limiting the spread of the contaminated aerosol throughout the room and replacing the contaminated air with cleansed air.

The present invention may also be used to increase the rate of air replenishment in the room. The volumetric flow of the room's ventilation system can be measured (m³/s) and given with respect to the volume of the room. This relationship may be defined as units of air changes per hour (ACH). Likewise, the volumetric flow of the device's filtering system can be measured (in m³/s) and formulated similarly in units of air changes per hour (ACH). Therefore the total air replenishment rate can be given by:

Air Replenishment Rate (ACH)=HVAC extraction (ACH)+Robot extraction (ACH)

The flowrate through the device serves to enhance the air replenishment rate, which is functionally equivalent to upgrading the heating, ventilation and air conditioning (HVAC) system to achieve a higher air removal rate.

Accordingly, the present invention will reduce the initial concentration of contaminant and improve airflow in the room, thus boosting the effectiveness of both mechanical room ventilation system and robot filtration system.

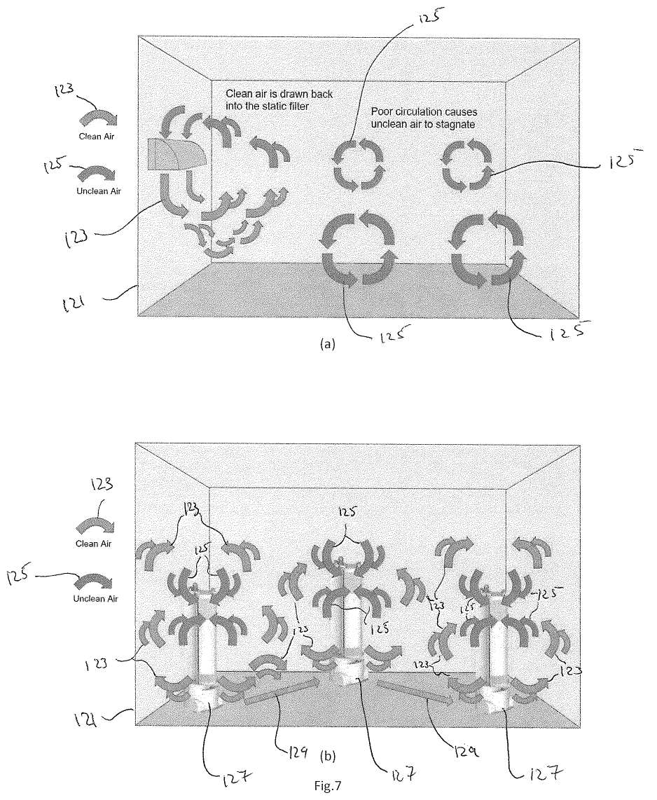

FIG. 7a is a schematic illustration of air flow in an enclosed space with a traditional vent and FIG. 7b is an illustration of air flow in an enclosed space using a device in accordance with the present invention. FIG. 7a shows an enclosed space 121 with arrows 123 and 125 representing the flow of clean air and unclean air respectively. In FIG. 7b, a device 127 in accordance with the present invention is shown along with the path 129 it moves through in the space 121, showing the device in use in three separate positions.

The effectiveness of the HVAC extraction system to replenish the air in the room with clean air is dependent on the quality of air mixing in the room, or the mixing factor. This is a measure of how clean air that enters the room mixes with the original air. In practice, most rooms contain areas of stagnation where air mixing is poor. When the device is introduced to the room, it improves air mixing in two ways:
1. Because it is mobile, it can draw in air from different parts of the room, including areas with high stagnation.
2. The directional exhaust allows the device to propel the filtered air away from the device. This has two benefits (i) it reduces the likelihood that previously filtered air is resampled by the robot, and (2) it allows the robot to actively promote mixing in parts of the room by creating a flow of air.

The present invention provides a system which determines where, when and how fast the device moves through the space, in an optimization strategy that takes into account different parameters of the room including the parameters of the HVAC system. The device's exhaust stream improves the overall air mixing in a space.

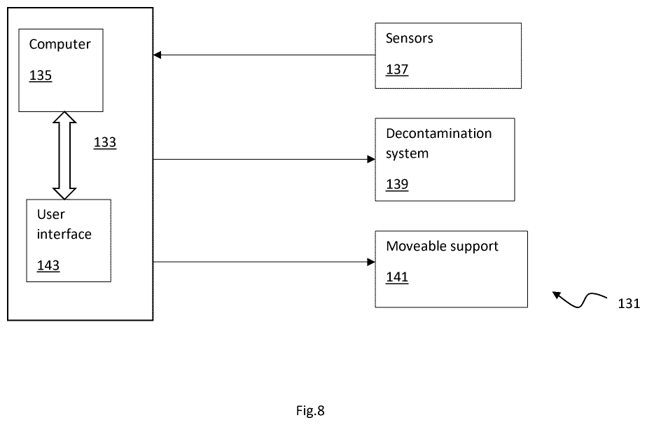

FIG. 8 is a schematic diagram 131 of an example of a control system in accordance with the present invention. FIG. 8 shows a control system 135 which comprises a computing means 135 which has a microprocessor and a memory containing software for implementing routines for receiving data, processing data and sending control signals to the peripheral devices. The user interface allows the user to select different operational modes and parameters for the device.

FIG. 8 shows sensors 137 which may include a depth camera, LIDAR or ultrasonic sensors which provide data on the position of the device. The decontamination system is connected to the control system to control the active components of the decontamination system such as the inlet aperture size and position, the outlet aperture orientation and the function of the radiation source. Data from the sensors 137 is processed and used to control the moveable support 141 to move the device to the appropriate position near an aerosol source or to move the device through an enclosed space to achieve effective decontamination/disinfection of the space.

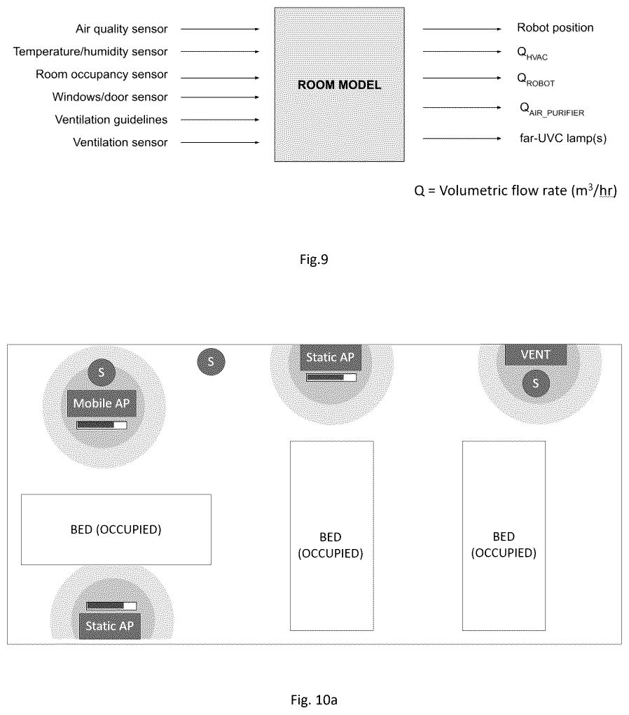

FIG. 9 shows a block diagram illustrating how environmental sensors, which may be located in the room or on the mobile/static air purification devices and other parameters are used as inputs to a room model, which is subsequently analysed to determine the optimal ventilation conditions, expressed in terms of parameters including the volumetric flow rate of air each air processor and the position of each mobile air processor in the room.

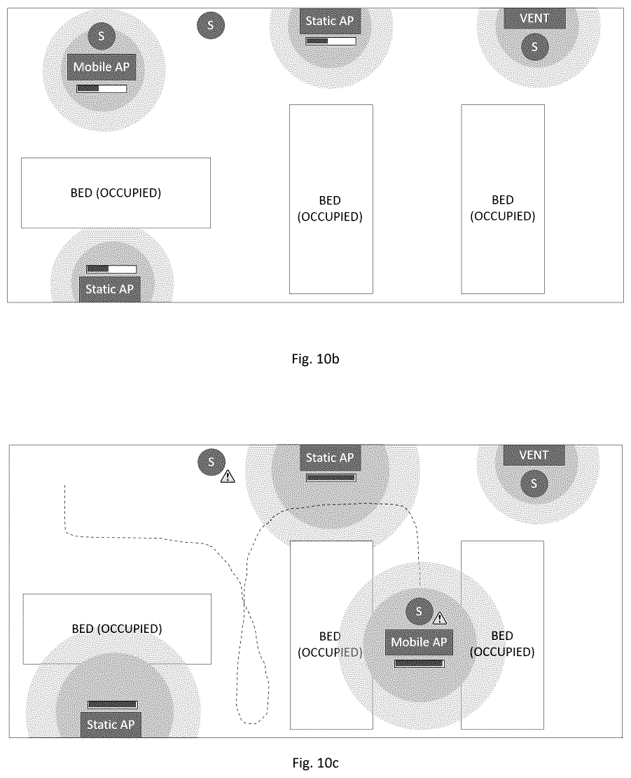

FIG. 10(a)-10(d) illustrates different configurations that the air decontamination system might adopt based on categorization of room model using data collected from a network of sensors in the local environment. The dark circular regions refer to the production of clean/disinfected air in the local area around the air purifier, and are intended for illustration only. The red bar adjacent to each air purifier illustrates the power setting used at that time. It is assumed in these illustrations that room ventilation cannot be controlled locally, and as such, this component only has a sensor that measures the flow through the vent.

In FIG. 10(a), airflow of mobile and static air purifiers (AP) located in room adjusted to achieve air replenishment requirements for the room considering airflow measured by sensor at the vent. This configuration is an example of a ‘normal’ operating state.

In FIG. 10(b), airflow of mobile and static air purifiers (AP) located in room adjusted to minimum achieve air replenishment requirements for the room considering airflow measured by sensor at the vent. This configuration is an example of an ‘ECO’ or ‘night’ mode.

In FIG. 10(c), airflow of mobile and static air purifiers (AP) located in room adjusted to exceed air replenishment requirements for the room considering airflow measured by sensor(S) at the vent. To maximise efficiency of air mixing, the mobile air purifier may reposition itself in different locations in the room. This may be initiated by a room sensor detecting an air quality risk.

In FIG. 10(d), airflow of mobile and static air purifiers (AP) located in room adjusted to ensure a clean air zone in the region of bed 2, while achieving air replenishment requirements for the room considering airflow measured by sensor at the vent. This configuration is an example of clean air zone state. The mobile air decontamination apparatus may be configured to reposition itself in different locations in the room to improve air mixing efficiency.

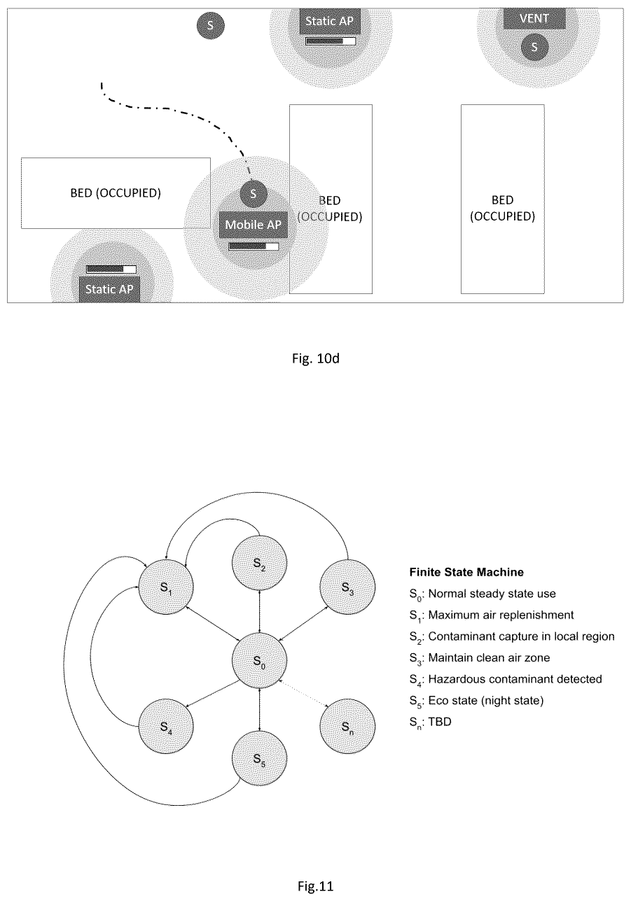

In FIG. 11, the different room model configurations are represented by a finite state machine. Each state corresponds with a different room model, which can be determined based on inference from the sensors. The operating parameters of each air purification device are determined by the current state of the system. The lines indicate how the system can transition between states. Transitions occur based on the room model changes into a new state. For example, if a sensor detects or measures a potentially hazardous substance, this may cause the state to transition from the stead-state (s0) to the hazardous state (s4), and then potentially to the maximum air replenishment state (s1).

FIG. 12 illustrates one configuration of the mobile air purifier embodiment, where the air outlet is directional and the clean air can be directed towards a target local area, such as a bed with a patient on it. Air intake is positioned towards the bottom of the apparatus, while air exhaust may be considered direction and is positioned towards the top of the apparatus.

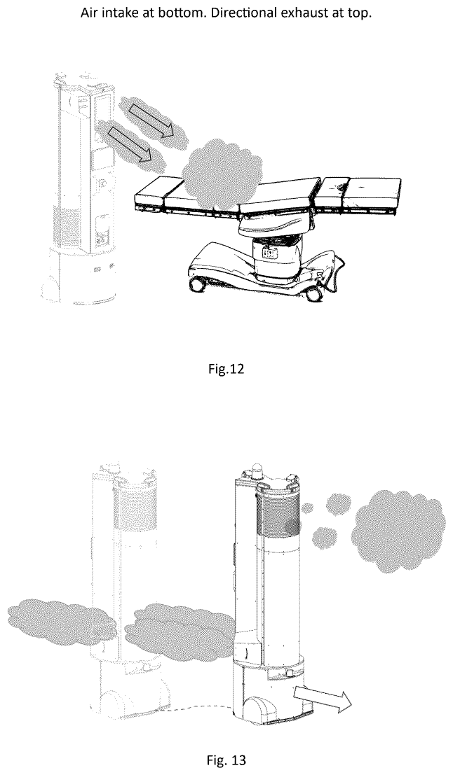

FIG. 13 illustrates one configuration of the mobile air purifier embodiment, where the air outlet is directional and clean air can be directed away from contaminated air in the local region of the air inlet.

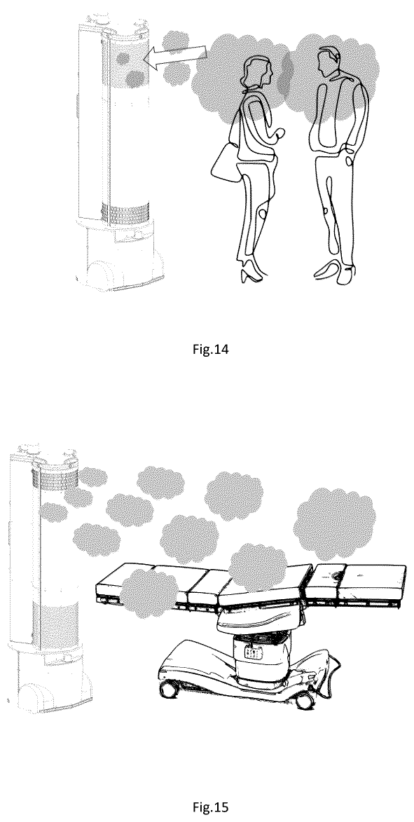

FIG. 14 illustrates one configuration of the mobile air purifier embodiment, where the air inlet is located at a height above the ground where contaminated particles may be present, and when positioned suitably close to a region comprising contaminated particles, can serve to extract and decontaminate these particles from the air.

FIG. 15 illustrates one configuration of the mobile air purifier embodiment, where the air outlet is turbulent and the clean air can be dispersed across a broad local area, such as a bed with a patient on it.

The present invention may comprise a mobile air decontamination device which has an array of sensors and associated computer hardware, firmware and software. The device may move autonomously or under manual control, using powered wheels or some other actuator configuration and comprise mechanisms for decontaminating air using an extraction fan connected in series with one or more mechanical filters or one or more radiation sources or a combination of mechanical filters and radiation sources for increasing levels of extraction and air mixing in the room.

The device is configured to cleanse air which has been contaminated by an aerosol generating procedure to increases the effective rate of air replenishment in a room and/or increases the rate of air mixing in the room through targeted positioning of its exhaust system.

The device may be configured in such a way that it serves as a form of extraction by limiting the spread of droplet or aerosolized microorganisms during an aerosol generating procedure.

Accordingly, the device may be used to increase number of air exchanges and improve mixing and/or to increase number of air exchanges and improve mixing and serve as a form of extraction.

The device may also have an exhaust mechanism which allows it to control the direction and exit velocity that the filtered air exits from the device, promoting increased air mixing in the region of the room where the exhaust is pointed.

In at least one embodiment, an algorithm is used to predict the optimal position and orientation of the device in the room relative to room furnishings and air vents to achieve high levels of air mixing, minimise the likelihood of re-sampled air, and/or redirect air towards vents towards/away from vents to optimize air flow.

The device may also have an extractor for extracting air from a specific direction relative to the device through an adaptable air inlet mechanisim that can be configured to extract air from local regions surrounding the device, or can be connected to a remotely located extractor.

The device may also have touch-sensitive handles that release an electronically controlled brake when grasped, and enable a human operator to manually reposition the device in the room.

The device may have a user interface displayed on that allows a human operator to set the desired air replenishment level, provide visual instructions on where to position the device in the room during the procedure, and how long

The invention will now be described with reference to specific examples. These are merely exemplary and for illustrative purposes only: they are not intended to be limiting in any way to the scope of the monopoly claimed or to the invention described. These examples constitute the best mode currently contemplated for practicing the invention.

Some of the embodiments of the invention described with reference to the drawings comprise a method implemented on computer apparatus and/or processes performed in a computer apparatus. However, the invention also extends to computer programs, particularly computer programs stored on or in a carrier adapted to bring the invention into practice. The program may be in the form of source code, object code, or a code intermediate source and object code, such as in partially compiled form or in any other form suitable for use in the implementation of the method according to the invention. The carrier may comprise a storage medium such as ROM, e.g. CD ROM, or magnetic recording medium, e.g. a memory stick or hard disk. The carrier may be an electrical or optical signal which may be transmitted via an electrical or an optical cable or by radio or other means.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. An air decontamination system that treats air containing contaminants, in one or more enclosed spaces, the air decontamination system comprising:
an enclosed space air decontamination system comprising:
one or more air quality sensors in an enclosed space of said one or more enclosed spaces;
one or more decontamination devices that treat air in the enclosed space; and
a communications interface which is operatively connectable to an air decontamination apparatus and the one or more air quality sensorscontaminant
wherein the air decontamination apparatus comprises:
a treatment system which comprises,
an extractor that draws air in from the enclosed space and
a decontamination system which receives the air from the extractor, the decontamination system being adapted to one or more of remove and neutralize contaminant aerosols in the air;
an outlet that expels treated air from the one or more decontamination devices;
a moveable support to which the treatment system is attached, which is moveable across a surface in an autonomous or manual manner; and
wherein the air decontamination system further comprises a system controller that controls operation of the air decontamination apparatus and the enclosed space air decontamination system.

2. The air decontamination system as claimed in claim 1, wherein, the system controller is located upon the air decontamination apparatus.

3. The air decontamination system as claimed in claim 1, wherein, the system controller is located within the enclosed space air decontamination system.

4. The air decontamination system as claimed in claim 1, wherein the air decontamination apparatus operates in a series of enclosed spaces by connecting to the communications interface in the enclosed space such that the system controller, when in communication with the communications interface, adapts the air decontamination apparatus in response to information received from the communications interface.

5. The air decontamination system as claimed in claim 1, wherein, the one or more air quality sensors comprise a network of sensors positioned in the enclosed space.

6. The air decontamination system as claimed in claim 1, wherein, the system controller uses a model of the enclosed space based on one or more of
dimensions of a room;
air vent location;
air flow; and
time of day to provide optimized air decontamination for the room while accounting for quality and energy efficiency.

7. The air decontamination system as claimed in claim 1, wherein the one or more air quality sensors comprise sensors which measure one or more of CO₂, particulate matter, Nitrous oxide, volatile organic compounds, bioburden, detection of specific pathogens, temperature or humidity.

8. The air decontamination system as claimed in claim 1, wherein, the one or more air quality sensors comprise sensors which measure one or more of occupancy, activity or other environmental parameters that influence need for air replenishment due to their effect on air quality.

9. The air decontamination system as claimed in claim 1, wherein, the enclosed space air decontamination system includes stationary air purifiers which comprise one or more of blower, air filter, UV decontaminator and chemical air disinfector.

10. The air decontamination system as claimed in claim 1, wherein, the air decontamination system integrates with a forced ventilation system in a building within which the one or more enclosed spaces are located to coordinate an effective means to maintain air quality.

11. The air decontamination system as claimed in claim 1, wherein, the enclosed space air decontamination system accounts for outdoor air quality, temperature, humidity.

12. The air decontamination system as claimed in claim 1, wherein, static air treatment units located within the enclosed space integrate with the air system controller.

13. The air decontamination system as claimed in claim 1, wherein, the air decontamination apparatus comprises at least one sensor that is configured to determine a position of the one or more decontamination devices in the enclosed space.

14. The air decontamination system as claimed in claim 1, wherein, the air decontamination apparatus comprises an inlet which is configurable to selectively draw air into the one or more decontamination devices from a predetermined specific space around the one or more decontamination devices.

15. The air decontamination system as claimed in claim 14, wherein, the inlet is extended from the mobile air decontamination device by means of a flexible tube, duct, or vanes and orientated to efficiently capture contaminated air from an aerosol generator.

16. The air decontamination system as claimed in claim 1, wherein, the outlet is controllable to set a direction in which the treated air is expelled from the one or more decontamination devices.

17. The air decontamination system as claimed in claim 1, wherein, the decontamination system comprises a mechanical filter.

18. The air decontamination system as claimed in claim 1, wherein, the decontamination system further comprises a radiation source.

19. The air decontamination system as claimed in claim 18, wherein, the radiation source is a UV source.

20. The air decontamination system as claimed in claim 1, wherein, the system controller comprises computing means that control the one or more air quality sensors and receives sensor data.

21. The air decontamination system as claimed in claim 1, wherein, the system controller comprises computer software that controls the air decontamination system.

22. The air decontamination system as claimed in claim 21, wherein, the computer software is configured to determine an optimal position of the air decontamination apparatus or multiple apparatuses in the enclosed space.

23. The air decontamination system as claimed in claim 21, wherein, the computer software is configured to identify one or more aerosol generating sources in the enclosed space.

24. The air decontamination system as claimed in claim 23, wherein, the computer software is configured to plan and execute a navigation path from a current location of the one or more decontamination devices to a location adjacent to the one or more aerosol generating sources.

25. The air decontamination system as claimed in claim 21, wherein, the computer software is configured to determine an optimal position in room to position the system controller and direct a flow of exhaust air to optimize airflow/air circulation.

26. The air decontamination system as claimed in claim 21, wherein, the computer software is configured to estimate a level of replenishment and provide a notification to an operator when a desired air replenishment has been obtained.

27. The air decontamination system as claimed in claim 1, wherein, the system controller controls a movement of the air decontamination apparatus in response to inputs from the one or more air quality sensors, occupancy sensors, activity sensors or navigation sensors.

28. The air decontamination system as claimed in claim 1, wherein, the moveable support comprises a base.

29. The air decontamination system as claimed in claim 1, wherein, the moveable support comprises wheels mounted to contact with a surface across which the air decontamination apparatus can travel.

30. The air decontamination system as claimed in claim 1, wherein, the air decontamination apparatus comprises a robotic system.

31. An air decontamination apparatus that treats air containing contaminant aerosols, in an enclosed space, the air decontamination apparatus comprising:
a treatment system that comprises
an extractor that draws air in from the enclosed space and
a decontamination system which receives the air from the extractor, the decontamination system being adapted to one or more of remove and neutralize the contaminant aerosols in the air;
an outlet that expels treated air from the decontamination system;
a moveable support to which the treatment system is attached, which is moveable across a surface; and
a system controller that controls operation of the treatment system and the moveable support wherein the air decontamination apparatus is operable with the an enclosed space decontamination system,
wherein the decontamination system comprises
one or more air quality sensors in the enclosed space;
one or more decontamination devices that treat air in the enclosed space; and
a communications interface which is operatively connectable to the air decontamination apparatus and the one or more air quality sensors.
